(19) United States
(12) Patent Application Publication
(10) Pub. No.: US 2026/0072001 A1
(43) Pub. Date: Mar. 12, 2026

(54) GAS SENSOR

(71) Applicant: TDK Corporation, Tokyo (JP)

(72) Inventor: Kei TANABE, Tokyo (JP)

(21) Appl. No.: 19/319,860

(22) Filed: Sep. 5, 2025

(30) Foreign Application Priority Data
Sep. 12, 2024 (JP) 2024-157975

(51) Int. Cl. G01N 30/86; G01N 30/66; G01N 33/00

(52) U.S. Cl. CPC G01N 30/8665; G01N 30/66; G01N 30/861; G01N 30/8651; G01N 33/004

(57) ABSTRACT

Disclosed herein is a gas sensor that includes: a sensor part configured to generate a gas detection signal according to a concentration of a gas to be measured; a differential amplifier configured to amplify a difference between the gas detection signal and a reference voltage to generate a differential signal; and a control circuit configured to generate an output signal indicating the concentration of the gas to be measured based on the differential signal. When the concentration of the gas to be measured falls below a threshold value corresponding to a level of the differential signal that is determined to be a concentration of the gas to be measured under normal conditions, the control circuit is configured to correct the reference voltage.

### CROSS-REFERENCE TO RELATED APPLICATION

This application claims the benefit of Japanese Patent Application No. 2024-157975, filed on Sep. 12, 2024, the entire disclosure of which is incorporated by reference herein.

### BACKGROUND OF THE ART

### Field of the Art

The present disclosure relates to a gas sensor and, more particularly, to a gas sensor capable of cancelling a negative drift.

### Description of Related Art

Japanese Patent No. 5563507 discloses a gas sensor of a type that compares a detection signal corresponding to the concentration of a gas to be measured with an offset voltage (reference voltage) using a differential amplifier to amplify the detection signal and calculates the concentration of the gas to be measured based on the amplified detection signal.

### SUMMARY

A gas sensor according to an aspect of the present disclosure includes: a sensor part configured to generate a gas detection signal according to a concentration of a gas to be measured; a differential amplifier configured to amplify a difference between the gas detection signal and a reference voltage to generate a differential signal; and a control circuit configured to generate an output signal indicating the concentration of the gas to be measured based on the differential signal. When the concentration of the gas to be measured falls below a threshold value corresponding to a level of the differential signal that is determined to be a concentration of the gas to be measured under normal conditions, the control circuit is configured to correct the reference voltage such that a level of the difference signal becomes equal to or more than the threshold value.

A gas sensor according to another aspect of the present disclosure includes: a sensor part configured to generate a gas detection signal according to a concentration of a gas to be measured; a differential amplifier configured to amplify a difference between the gas detection signal and a reference voltage to generate a differential signal; and a control circuit configured to generate an output signal indicating the concentration of the gas to be measured based on the differential signal. When the concentration of the gas to be measured falls below a threshold value corresponding to a level of the differential signal that is determined to be a concentration of the gas to be measured under normal conditions, the control circuit is configured to correct the reference voltage such that a level of the difference signal becomes equal to or more than the threshold value.

### BRIEF DESCRIPTION OF THE DRAWINGS

The above features and advantages of the present disclosure will be more apparent from the following description of some embodiments taken in conjunction with the accompanying drawings, in which:

FIG. 1 is a circuit diagram illustrating the configuration of a gas sensor 100 according to an embodiment of the technology described herein;

FIGS. 2A and 2B are circuit examples of the reference voltage generation circuit 32;

FIG. 3 is a flowchart for explaining for the environmental temperature measurement operation of the gas sensor 100;

FIG. 4 is a flowchart for explaining a first example of the gas concentration measurement operation of the gas sensor 100;

FIG. 5 is a flowchart for explaining a modification of the first example of the gas concentration measurement operation of the gas sensor 100;

FIG. 6 is a flowchart for explaining a second example of the gas concentration measurement operation of the gas sensor 100;

FIGS. 7A and 7B are schematic graphs for explaining a first effect brought about by the gas sensor 100;

FIGS. 8A and 8B are schematic graphs for explaining a second effect brought about by the gas sensor 100;

FIGS. 9A to 9D are schematic graphs for explaining an example of the operation of the gas sensor 100 when the concentration of CO₂ gas in measurement atmosphere changes;

FIG. 10 is a circuit diagram illustrating the configuration of a gas sensor 100a according to a first modification;

FIG. 11 is a circuit diagram illustrating the configuration of a gas sensor 100b according to a second modification; and

FIG. 12 is a circuit diagram illustrating the configuration of a gas sensor 100c according to a third modification.

### DETAILED DESCRIPTION OF THE EMBODIMENTS

In the gas sensor described in Japanese Patent No. 5563507, a calculation error will occur in the concentration of the gas to be measured due to the influence of a drift caused by a temporal change.

The present disclosure describes an improved gas sensor capable of cancelling the influence of a drift.

Some embodiments of the present disclosure will be explained below in detail with reference to the accompanying drawings.

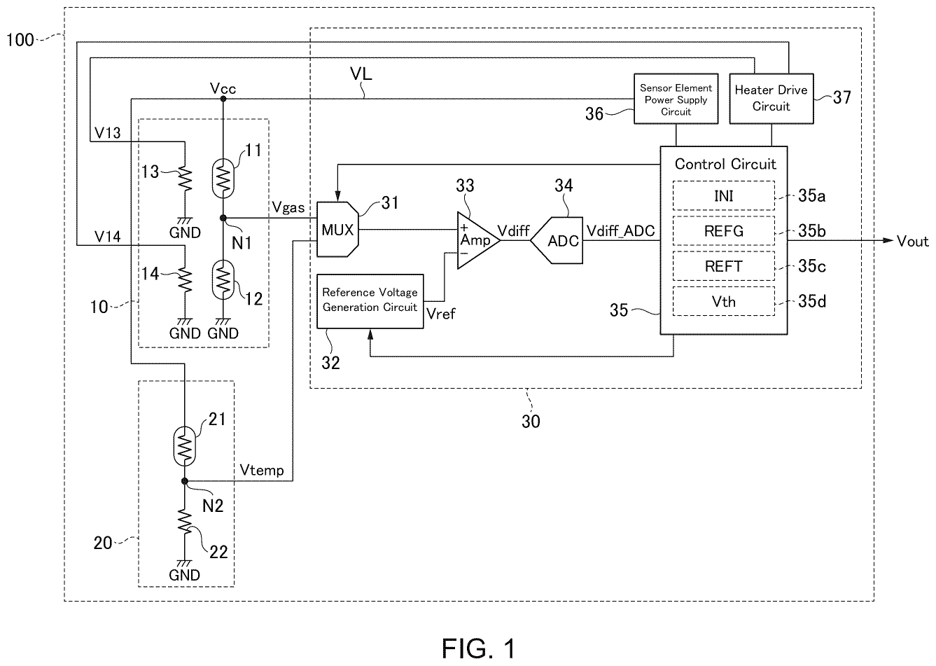

FIG. 1 is a circuit diagram illustrating the configuration of a gas sensor 100 according to an embodiment of the technology described herein.

As described in FIG. 1, the gas sensor 100 according to the present embodiment includes a sensor part 10 that generates a gas detection signal Vgas according to the concentration of a gas to be measured, a temperature sensor 20 that generates a temperature signal Vtemp according to an environmental temperature, and a signal processing circuit 30. Although not particularly limited, the gas sensor 100 according to the present embodiment is a heat-conduction type gas sensor for detecting the concentration of CO₂ gas in measurement atmosphere.

The sensor part 10 includes thermistors 11 and 12 connected in series between a power supply line VL and a ground GND and heaters 13 and 14 for heating the thermistors 11 and 12, respectively. The gas detection signal Vgas output from the sensor part 10 appears at a node N1 between the thermistors 11 and 12. The thermistor 11 is a temperature-sensitive element for detection, and the thermistor 12 is a temperature-sensitive element for reference. The thermistors 11 and 12 are each a resistor whose resistance value changes with temperature. The thermistors 11 and 12 and a thermistor 21 to be described later may be made of, for example, vanadium oxide, amorphous silicon, polycrystalline silicon, an oxide with a spinel crystal structure containing manganese, titanium oxide, or yttrium-barium-copper oxide.

When CO₂ gas is present in measurement atmosphere in a state where the thermistor 11 as the detection temperature-sensitive element is heated to a temperature range of 100° C. to 230° C. (e.g., around 150° C.) which is highly sensitive to the heat dissipation characteristics of the CO₂ gas, thermistor 11 change according to the concentration of CO₂ gas. This change appears as a change in the temperature of the thermistor 11, i.e., a change in the resistance value thereof. For example, CO₂ gas is lower in heat dissipation than air, so that the temperature of the thermistor 11 increases as the concentration of CO₂ gas becomes high. Here, assume that heating is performed such that the temperature of the thermistor 11 becomes 150° C. when the concentration of CO₂ gas in measurement atmosphere indicates a concentration value (e.g., 400 ppm) of CO₂ gas under ordinary atmospheric environment. In this case, when heating is performed such that the temperature of the thermistor 11 becomes 150° C. in a state where the concentration of CO₂ gas present in measurement atmosphere exceeds the concentration value under ordinary atmospheric environment, the temperature of the thermistor 11 increases with an increase in the concentration of CO₂ gas and exceeds 150° C. As a result, the resistance value of the thermistor 11 is lowered as the concentration of CO₂ gas in measurement atmosphere increases.

On the other hand, even when CO₂ gas is present in measurement atmosphere in a state where the thermistor 12 as the reference temperature-sensitive element is heated to a temperature range of 250° C. to 450° C. (e.g., around 300° C.) which is lowly sensitive to CO₂ gas, the heat dissipation characteristics of the thermistor 12 hardly change according to the concentration of CO₂ gas, and the temperature thereof also hardly changes. Accordingly, a CO₂ gas concentration-dependent change in the resistance value of the thermistor 12 heated to around 300° C. is sufficiently smaller than a CO₂ gas concentration-dependent change in the resistance value of the thermistor 11 heated to around 150° C. There may be almost no CO₂ gas concentration-dependent change in the resistance value of the thermistor 12 heated to around 300° C. As a result, when the thermistors 11 and 12 are heated to around 150° C. and around 300° C., respectively (when heating is performed such that temperatures of the thermistors 11 and 12 become 150° C. and 300° C., respectively, in a state where the concentration of CO₂ gas in measurement atmosphere indicates a concentration value under ordinary atmospheric environment), the gas detection signal Vgas corresponding to the concentration of CO₂ gas in measurement atmosphere appears at the node N1 between the thermistors 11 and 12. On the other hand, even when there is contained, in measurement atmosphere, another gas that brings about no significant difference between the heat dissipation characteristics of the thermistor 11 exhibited when it is heated to around 150° C. and those of the thermistor 12 exhibited when it is heated to around 300° C., the concentration of this gas could hardly affect the gas detection signal Vgas. This allows the sensor part 10 to selectively detect the concentration of CO₂ gas.

The temperature sensor 20 includes a thermistor 21 and a resistor 22 which are connected in series between the power supply line VL and ground GND. The temperature signal Vtemp of the temperature sensor 20 appears at a node N2 between the thermistor 21 and the resistor 22. The temperature sensor 20 detects an environmental temperature. The environmental temperature is a temperature in measurement atmosphere. The temperature sensor 20 may be designed so as not to be affected or so as to be hardly affected by heating by, for example, the heaters 13 and 14.

The signal processing circuit 30 includes a multiplexer 31, a reference voltage generation circuit 32, a differential amplifier 33, an AD converter (ADC) 34, a control circuit 35, a sensor element power supply circuit 36, and a heater drive circuit 37.

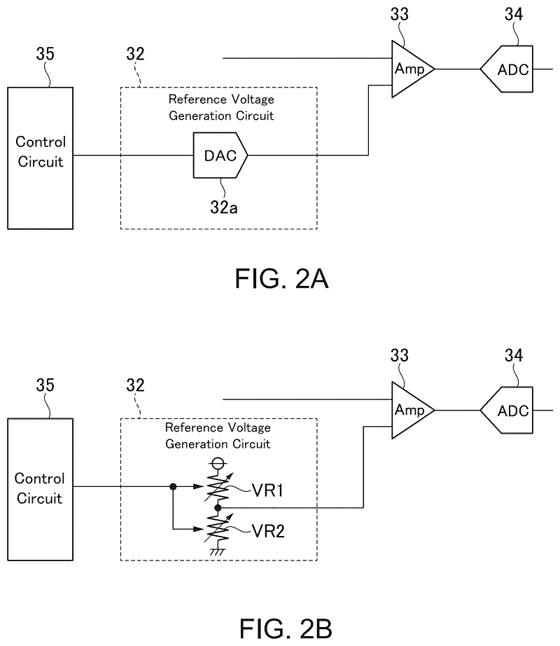

The multiplexer 31 supplies either the gas detection signal Vgas or the temperature signal Vtemp to the differential amplifier 33 under the control of the control circuit 35. The differential amplifier 33 amplifies the difference (potential difference) between the level of one of the gas detection signal Vgas and temperature signal Vtemp and the level of a reference voltage Vref generated by the reference voltage generation circuit 32 to generate a differential signal Vdiff. Alternatively, the differential amplifier 33 may amplify the difference (potential difference) between the level of one of the gas detection signal Vgas and temperature signal Vtemp and the level of a reference voltage Vref with a desired amplification factor of ×1 or more, or less than ×1 to generate the differential signal Vdiff. The reference voltage generation circuit 32 may be configured as a DA converter (DAC) 32a that DA-converts a digital value output from the control circuit 35 as illustrated in FIG. 2A or may be configured by including variable resistors VR1 and VR2 whose resistance values are controlled by the control circuit 35 as illustrated in FIG. 2B. In either case, the level of the reference voltage Vref is determined by an initial value INI stored in a memory 35a included in the control circuit 35, a set value REFG stored in a memory 35b included in the control circuit 35, or a set value REFT stored in a memory 35c included in the control circuit 35. The initial value INI may be a fixed or switchable value. Further, as described later, the set value REFG is updated at the time of a gas concentration measurement operation.

Although the polarity of the differential amplifier 33 is not particularity limited, in the example illustrated in FIG. 1, the gas detection signal Vgas or temperature signal Vtemp is supplied to a non-inversion input terminal (+), and the reference voltage Vref is supplied to an inversion input terminal (−). In this case, the higher the concentration of CO₂ gas in measurement atmosphere is, the higher the level of the differential signal Vdiff becomes.

The differential signal Vdiff output from the differential amplifier 33 is input to the AD converter 34. The AD converter 34 AD-converts the differential signal Vdiff to generate a differential signal Vdiff_ADC having a digital value and supplies it to the control circuit 35.

The control circuit 35 calculates the concentration of CO₂ gas which is a gas to be measured based on the differential signal Vdiff_ADC and generates an output signal Vout indicating the concentration of CO₂ gas. The control circuit 35 may calculate the concentration of CO₂ gas using a calculation formula set therein. Further, the control circuit 35 supplies a power supply voltage Vcc to the sensor part 10 and temperature sensor 20 through the sensor element power supply circuit 36 and controls, through the heater drive circuit 37, the levels of the heater voltages V13 and V14 supplied to the heaters 13 and 14, respectively.

The control circuit 35 corrects the heater voltages V13 and V14 in accordance with the differential signal Vdiff obtained as a result of amplification of the difference between the temperature signal Vtemp and the reference voltage Vref. For example, when the concentration of CO₂ gas in measurement atmosphere indicates a concentration value (e.g., 400 ppm) of CO₂ gas under ordinary atmospheric environment, the control circuit 35 corrects the heater voltages V13 and V14 such that the temperatures of the thermistors 11 and 12 become to 150° C. and 300° C., respectively, irrespective of the environmental temperature as a result of heating for a predetermined period of time using the heaters 13 and 14. That is, the control circuit 35 changes the levels of the heater voltages V13 and V14 in accordance with the temperature signal Vtemp (to be exact, the differential signal Vdiff_ADC obtained by AD-converting the difference between the temperature signal Vtemp and the reference voltage Vref) to change powers to be applied to the heaters 13 and 14, respectively, thereby changing the heating amounts of the heaters 13 and 14. The level of the reference voltage Vref at the time of an environmental temperature measurement operation is determined by the set value REFT stored in the memory 35c included in the control circuit 35. The set value REFT may be a fixed value.

Further, when the level of the differential signal Vdiff_ADC obtained by AD-converting the difference between the gas detection signal Vgas and the reference voltage Vref falls below a threshold value Vth stored in the memory 35d included in the control circuit 35, the control circuit 35 changes the set value REFG set in the memory 35b such that the level of the differential signal Vdiff_ADC becomes equal to or more than the threshold value Vth or becomes close thereto, thereby correcting the level of the reference voltage Vref. When the level of the differential signal Vdiff_ADC is equal to the threshold value Vth, the control circuit 35 sets the level of the output signal Vout to a level corresponding to that obtained when the concentration of CO₂ gas which is a gas to be measured indicates a concentration value (e.g., 400 ppm) of CO₂ gas under ordinary atmospheric environment. In other words, the threshold value Vth corresponds to the level of the differential signal Vdiff_ADC to be referred to when the control circuit 35 determines that the concentration of CO₂ gas which is a gas to be measured indicates a concentration value (e.g., 400 ppm) of CO₂ gas under ordinary atmospheric environment.

The following describes in more detail the operation of the gas sensor 100 according to the present embodiment.

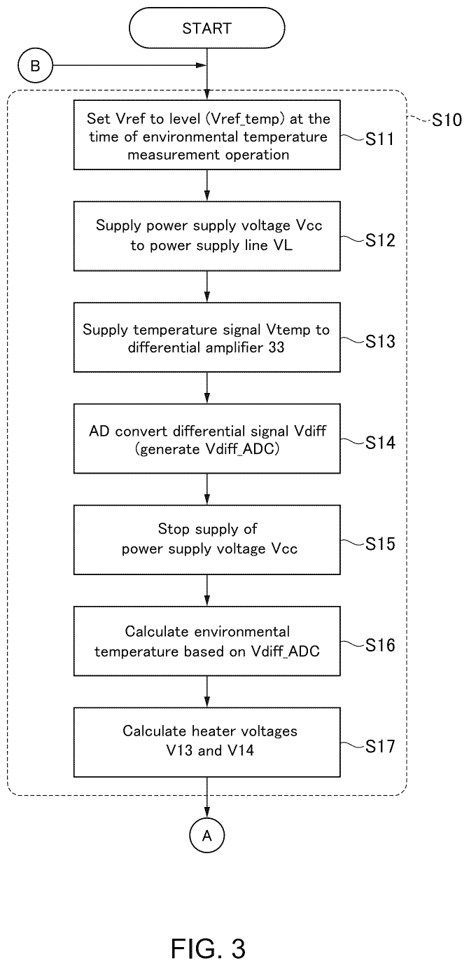

FIG. 3 is a flowchart for explaining for the environmental temperature measurement operation of the gas sensor 100.

The gas sensor 100 starts an environmental temperature measurement operation S10, and the control circuit 35 reads out the set value REFT stored in the memory 35c and controls the reference voltage generation circuit 32 based on the read out set value REFT, thereby setting the level of the reference voltage Vref to a level (Vref_temp) at the time of the environmental temperature measurement operation (step S11). Subsequently, the control circuit 35 controls the sensor element power supply circuit 36 to supply the power supply voltage Vcc to the power supply line VL (step S12). As a result, the power supply voltage Vcc is applied to the temperature sensor 20, and the temperature signal Vtemp corresponding to the environmental temperature appears at the node N2.

Then, the control circuit 35 controls the multiplexer 31 to supply the temperature signal Vtemp to the differential amplifier 33 (step S13). As a result, the differential signal Vdiff obtained as a result of amplification of the potential difference between the temperature signal Vtemp and the reference voltage Vref is output from the differential amplifier 33. In this state, the AD converter 34 performs AD conversion and supplies the obtained differential Vdiff_ADC having a digital value to the control circuit 35 (step S14). Thereafter, the control circuit 35 controls the sensor element power supply circuit 36 to stop supply of the power supply voltage (step S15) and calculates the environmental temperature based on the differential signal Vdiff_ADC (step S16). Then, the control circuit 35 calculates the levels of the heater voltages V13 and V14 according to the calculated environmental temperature (step S17).

After completion of the environmental temperature measurement operation S10, the gas concentration measurement operation is executed.

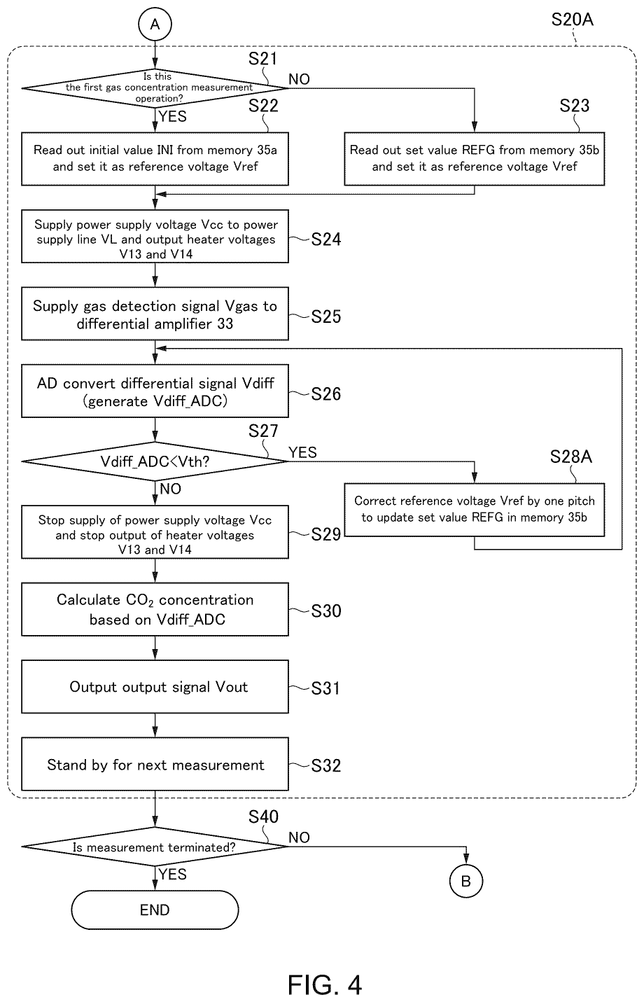

FIG. 4 is a flowchart for explaining a first example of the gas concentration measurement operation of the gas sensor 100.

The gas sensor 100 starts a gas concentration measurement operation S20A. When this gas concentration measurement operation is the first gas concentration measurement operation (YES in step S21), the control circuit 35 reads out the initial value INI set in the memory 35a and controls the reference voltage generation circuit 32 based on the read-out initial value INI, thereby setting the level of the reference voltage Vref to the initial value INI of the level (Vref_gas) at the time of the gas concentration measurement operation (step S22). The initial value INI is used as the initial value of the set value REFG set in the memory 35b. On the other hand, when the gas concentration measurement operation is the second or subsequent gas concentration measurement operations (NO in step S21), the set value REFG set in the memory 35b is read out, and the level of the reference voltage Vref is set based on the read-out set value REFG (step S23).

Then, the control circuit 35 controls the sensor element power supply circuit 36 to supply the power supply voltage Vcc to the power supply line VL and controls the heater drive circuit 37 to supply the heater voltages V13 and V14 to the heaters 13 and 14, respectively (step S24). As a result, the power supply voltage Vcc is applied to the sensor part 10, and the gas detection signal Vgas corresponding to the concentration of CO₂ gas in measurement atmosphere appears at the node N1.

Then, the control circuit 35 controls the multiplexer 31 to supply the gas detection signal Vgas to the differential amplifier 33 (step S25). As a result, the differential signal Vdiff obtained as a result of amplification of the potential difference between the gas detection signal Vgas and the reference voltage Vref is output from the differential amplifier 33. In this state, the AD converter 34 performs AD conversion and supplies the obtained differential signal Vdiff_ADC having a digital value to the control circuit 35 (step S26).

Then, the control circuit 35 compares the level of the differential signal Vdiff_ADC and the threshold value Vth set in the memory 35d (step S27). As described above, the threshold value Vth corresponds to the level of the differential signal Vdiff_ADC to be referred to when the control circuit 35 determines that the concentration of CO₂ gas which is a gas to be measured indicates a concentration value (e.g., 400 ppm) of CO₂ gas under ordinary atmospheric environment. That is, when the concentration of CO₂ gas under ordinary atmospheric environment is 400 ppm, the concentration of CO₂ gas in measurement atmosphere does not usually become less than 400 ppm, and thus the concentration of CO₂ gas indicated by the differential signal Vdiff_ADC should be 400 ppm or more unless a negative drift occurs in the sensor part 10, so that the level of the differential signal Vdiff_ADC becomes equal to or more than the ordinary threshold value Vth. The negative drift refers to a phenomenon that the level of gas detection signal Vgas is lowered with time even when an actual gas concentration is constant.

When the level of the differential signal Vdiff_ADC is equal to or more than the threshold value Vth (NO in step S27), the control circuit 35 controls the sensor element power supply circuit 36 to stop supply of the power supply voltage Vcc, controls the heater drive circuit 37 to stop supply of the heater voltages V13 and V14 (step S29), and calculates the concentration of CO₂ gas based on the differential signal Vdiff_ADC (step S30). The calculated concentration of CO₂ gas is externally output as the output signal Vout. Thereafter, the control circuit 35 stands by for the next measurement operation (step S32). When the level of the differential signal Vdiff_ADC is equal to or more than the threshold value Vth in step S27, the value of the reference voltage Vref is retained, and thus the set value REFG in the memory 35b is retained. In this case, when this operation is the first gas concentration measurement operation, the value of the initial value INI is written into the memory 35b as the set value REFG for update.

On the other hand, when the level of the differential signal Vdiff_ADC falls below the threshold value Vth in step S27 (YES in step S27), the control circuit 35 corrects the set value REFG in the memory 35b so as to lower the level of the reference voltage Vref (step S28A). As described above, the threshold value Vth corresponds to the level of the differential signal Vdiff_ADC to be referred to when the control circuit 35 determines that the concentration of CO₂ gas which is a gas to be measured indicates a concentration value (e.g., 400 ppm) of CO₂ gas under ordinary atmospheric environment, so that the fact that the level of the differential signal Vdiff_ADC is below the threshold value Vth means that a negative drift has occurred in the sensor part 10. In step S28A, the control circuit 35 corrects the reference voltage Vref so as to cancel the negative drift.

The amount of correction for the reference voltage Vref may be a correctable minimum pitch. For example, when the reference voltage generation circuit 32 illustrated in FIG. 2A is used, it is possible to lower the level of the reference voltage Vref by one pitch by incrementing a digital value to be supplied to the DA converter 32a by one bit. When the reference voltage Vref is thus corrected and lowered in level, the level of the differential signal Vdiff also changes (increases). The control circuit 35 updates the set value REFG in the memory 35b to the value of the reference voltage Vref after correction in association with the correction of the reference voltage Vref. After the reference voltage Vref is corrected, the flow returns to step S26, where the AD converter 34 performs AD conversion and supplies the obtained differential signal Vdiff_ADC to the control circuit 35.

The above operation is repeatedly executed until the level of the differential signal Vdiff_ADC becomes equal to or more than the threshold value Vth. When the level of the differential signal Vdiff_ADC is equal to or more than the threshold value Vth (NO in step S27), step S29 and subsequent steps are executed. The update of the set value REFG in the memory 35b may be executed based on the level of the final reference voltage Vref when NO is determined in step S27, instead of being executed every time in step S28A. Thus, even when step S28A is repeatedly executed for several times, the update of the set value REFG in the memory 35b can be completed in one operation.

Thus, the gas concentration measurement operation S20A is terminated. When a series of the measurements is not terminated (NO in step S40), the flow returns to the environmental temperature measurement operation S10 illustrated in FIG. 3. In the second and subsequent gas concentration measurement operations S20A, the set value REFG set in the memory 35b is read out in step S23, and the level of the reference voltage Vref is set based on the read-out set value REFG. For example, the latest set value REFG (value of the reference voltage Vref upon generation of the differential signal used in the previous gas concentration calculation) retained in step S27 or updated in step S28 is used. That is, the control circuit 35 updates the set value REFG every time the reference voltage Vref is corrected, so that, in the second and subsequent gas concentration measurement operations S20A, the level of the reference voltage Vref is set based on the updated latest set value REFG. When the measurement is terminated (YES in step S40), a series of the measurements is terminated.

Thus, in the gas concentration measurement operation according to the first example, when the level of the differential signal Vdiff_ADC falls below the threshold value Vth (YES in step S27), the reference voltage Vref is corrected stepwise until the level of the differential signal Vdiff_ADC becomes equal to or more than the threshold value Vth (step S28A), so that it is possible to cancel a negative drift that has occurred in the sensor part 10. In addition, when the amount of correction for the reference voltage Vref per time is made constant, complicated calculation is not required.

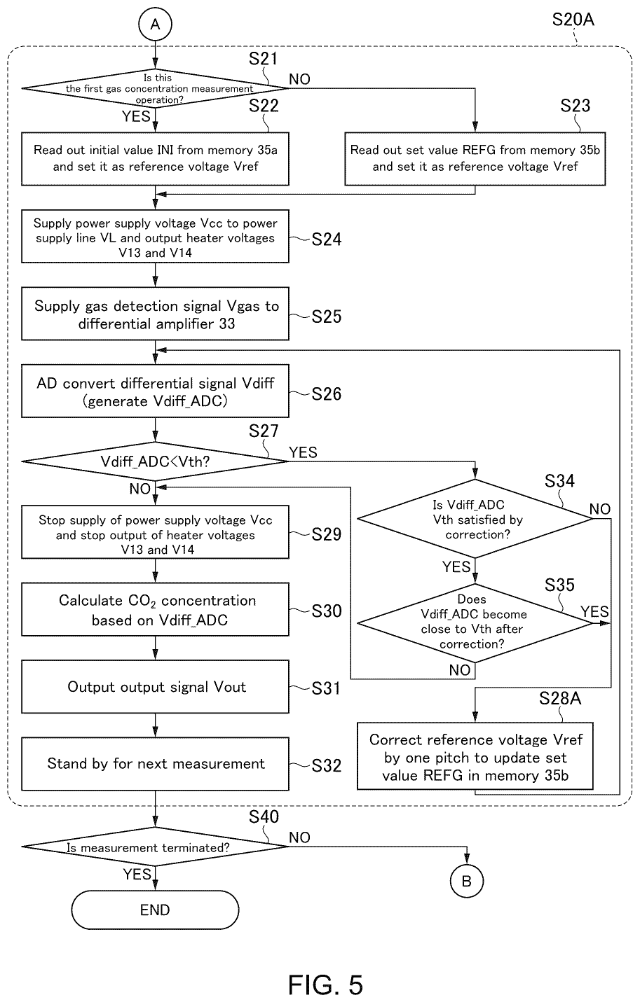

FIG. 5 is a flowchart for explaining a modification of the first example of the gas concentration measurement operation of the gas sensor 100.

In the modification illustrated in FIG. 5, steps S34 and S35 are added between step S27 and step S28A. In step S34, it is determined whether the level of the differential signal Vdiff_ADC after correction is equal to or more than the threshold value Vth under the assumption that the level of the reference voltage Vref is lowered by one pitch in step S28A. When it is determined that the level of the differential signal Vdiff_ADC after correction is less than the threshold value Vth, the flow proceeds to step S28A, the level of the reference voltage Vref is actually lowered by one pitch. On the other hand, when it is determined that the level of the differential signal Vdiff_ADC after correction is equal to or more than the threshold value Vth, the flow proceeds to step S35.

In step S35, it is determined whether the level of the differential signal Vdiff_ADC becomes closer to the threshold value Vth under the assumption that the level of the reference voltage Vref is lowered by one pitch in step S28A. That is, it is determined whether the absolute value of the difference between the level of the differential signal Vdiff_ADC after correction and the threshold value Vth becomes smaller than the absolute value of the difference between the level of the differential signal Vdiff_ADC before correction and the threshold value Vth. When it is determined that the level of the differential signal Vdiff_ADC after correction becomes closer to the threshold value Vth, the flow proceeds to step S28A, where the level of the reference voltage Vref is actually lowered by one pitch. On the other hand, when it is determined that the level of the differential signal Vdiff_ADC after correction becomes more distant from the threshold value Vth, the differential signal Vdiff_ADC is not corrected, and the flow proceeds to step S29.

According to the modification illustrated in FIG. 5, even when the level of the differential signal Vdiff_ADC after correction is equal to or more than the threshold value Vth, correction is not performed any longer for the differential signal Vdiff_ADC if the level of the differential signal Vdiff_ADC after correction becomes more distant from the threshold value Vth. As a result, the level of the differential signal Vdiff_ADC after correction certainly becomes closer to the threshold value Vth than the level of the differential signal Vdiff_ADC before correction, thus making it possible to prevent a measurement error which may be caused due to excessive correction of the differential signal Vdiff_ADC. For example, assume that the concentration of CO₂ gas indicated by the threshold value Vth is 400 ppm and that the concentration of CO₂ gas indicated by the current differential signal Vdiff_ADC is 390 ppm. In this case, when the correction pitch is 30 ppm, the concentration of CO₂ gas indicated by the differential signal Vdiff_ADC after correction is 420 ppm, and the absolute value of the difference between the level of the differential signal Vdiff_ADC and the threshold value Vth increases from 10 ppm to 20 ppm. Thus, such excessive correction can be avoided.

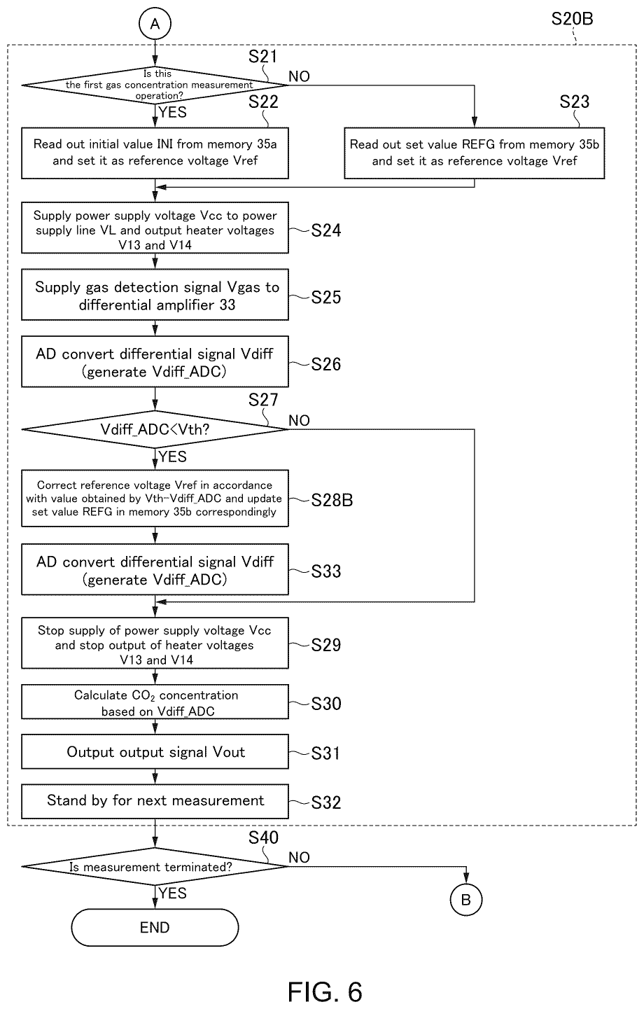

FIG. 6 is a flowchart for explaining a second example of the gas concentration measurement operation of the gas sensor 100.

In a gas concentration measurement operation S20B according to the second example illustrated in FIG. 6, operations from steps S21 to S27 are the same as those in the gas concentration measurement operation S20A according to the first example illustrated in FIG. 4. When the level of the differential signal Vdiff_ADC is equal to or more than the threshold value Vth (NO in step S27), steps S29 to S32 are executed as in the gas concentration measurement operation S20A according to the first example illustrated in FIG. 4.

On the other hand, when it is determined in step S27 that the level of the differential signal Vdiff_ADC falls below the threshold value Vth (YES in step S27), the control circuit 35 lowers the level of the reference voltage Vref in accordance with the difference between the level of the differential signal Vdiff_ADC and the threshold value Vth so as to reduce this difference, i.e., such that the level of the reference voltage Vref becomes close to the threshold value Vth and updates the set value REFG in the memory 35b to the value of the reference voltage Vref after correction (step S28B). In this case, the reference voltage Vref may be corrected such that the difference becomes equal to or more than 0, i.e., the level of the differential signal Vdiff_ADC becomes equal to or more than the threshold value Vth or such that the difference becomes less than 0, i.e., the level of the differential signal Vdiff_ADC becomes less than the threshold value Vth. For example, assuming that the gain of the differential amplifier 33 is G, the reference voltage Vref is corrected such that the level thereof is lowered by (Vth−Vamp_ADC)/G, and the set value REFG is updated correspondingly.

After the reference voltage Vref is thus corrected, the AD converter 34 performs AD conversion again and supplies the obtained differential signal Vdiff_ADC to the control circuit 35 (step S33). In step S33, the reference voltage Vref has already been corrected, so that the level of the differential signal Vdiff_ADC becomes closer to the threshold value Vth. Thereafter, steps S29 to S32 are executed.

As described above, in the gas concentration measurement operation S20B according to the second example, when the level of the differential signal Vdiff_ADC falls below the threshold value Vth (YES in step S27), the reference voltage Vref is corrected using a correction amount corresponding to the difference between the differential signal Vdiff_ADC and the threshold value Vth (step S28B), thereby making it possible to cancel a negative drift occurring in the sensor part 10 in one operation.

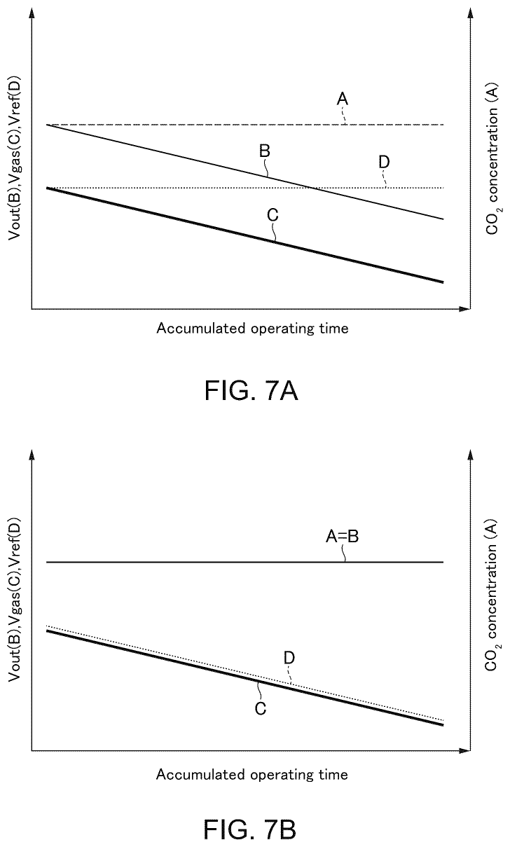

FIGS. 7A and 7B are schematic graphs for explaining a first effect brought about by the gas sensor 100. FIG. 7A illustrates an example in which cancellation (correction of the reference voltage Vref) of a negative drift is not performed, and FIG. 7B illustrates an example in which cancellation (correction of the reference voltage Vref) of a negative drift is performed. In FIGS. 7A and 7B, a sign A denotes the actual concentration (constant value) of CO₂ gas, a sign B denotes the concentration of CO₂ gas indicated by the output signal Vout, a sign C denotes the level of the gas detection signal Vgas, and a sign D denotes the level of the reference voltage Vref.

As can be seen from FIG. 7A in which the negative drift cancellation (correction of the reference voltage Vref) is not performed (D=constant), in a state where a negative drift has occurred as denoted by the sign C, the level of the gas detection signal Vgas is lowered with time even when the actual concentration A of CO₂ gas is constant, with the result that the CO₂ gas concentration indicated by the output signal Vout is also lowered as denoted by the sign B. On the other hand, as illustrated in FIG. 7B, in the present embodiment in which the negative drift cancellation (correction of reference voltage Vref) is performed, when the level of the gas detection signal Vgas is lowered with time due to a negative drift, the level of the reference voltage Vref is also lowered following this, so that the output signal Vout can indicate the concentration of CO₂ gas in which the influence of a negative drift has been canceled.

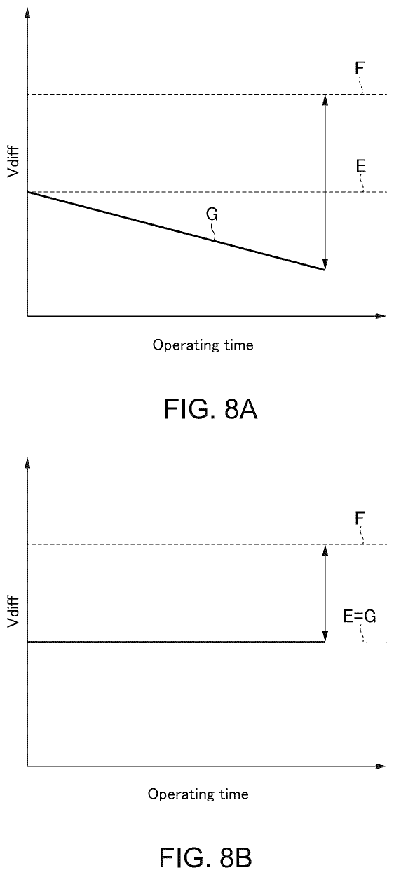

FIGS. 8A and 8B are schematic graphs for explaining a second effect brought about by the gas sensor 100. FIG. 8A illustrates an example in which cancellation (correction of the reference voltage Vref) of a negative drift is not performed, and FIG. 8B illustrates an example in which cancellation (correction of the reference voltage Vref) of a negative drift is performed. In FIGS. 8A and 8B, a sign E denotes a correct level (level obtained when no drift is present) of the differential signal Vdiff to be obtained when the concentration of CO₂ gas in measurement atmosphere indicates a concentration value (e.g., 400 ppm) of CO₂ gas under ordinary atmospheric environment, a sign F denotes a correct level (level obtained when no drift is present) of the differential signal Vdiff to be obtained when the concentration of CO₂ gas in measurement atmosphere indicates a concentration value (e.g., 5000 ppm) higher than the concentration of CO₂ gas under ordinary atmospheric environment, and a sign G denotes the level of the differential signal Vdiff to be actually obtained when the concentration of CO₂ gas in measurement atmosphere indicates a concentration value (e.g., 400 ppm) of CO₂ gas under ordinary atmospheric environment.

As can be seen from FIG. 8A in which the negative drift cancellation (correction of the reference voltage Vref) is not performed, in a state where a negative drift has occurred as denoted by the sign G, the level of the differential signal Vdiff is lowered with time even when the concentration of CO₂ gas in measurement atmosphere is constant at a concentration value (e.g., 400 ppm) of CO₂ gas under ordinary atmospheric environment. As a result, the level difference between the signs F and G increases with time, so that a negative drift component enters in the dynamic range of the differential amplifier 33, which substantially reduces a part of the dynamic range of the differential amplifier 33 that can be used for detection of the concentration of CO₂ gas. On the other hand, in the present embodiment in which the negative drift cancellation (correction of reference voltage Vref) is performed, the level difference between the signs F and G does not change as illustrated in FIG. 8B, so that it is possible to sufficiently ensure a part of the dynamic range of the differential amplifier 33 that can be used for detection of the concentration of CO₂ gas. Further, for example, in the embodiment illustrated in FIG. 1, the AD converter 34 is provided in the rear stage of the differential amplifier 33, and the differential signal Vdiff output from the differential amplifier 33 is input to the AD converter 34. In such a case, when the negative drift cancellation (correction of reference voltage Vref) is not performed, the level difference between the signs F and G increases with time as illustrated in FIG. 8A, thus requiring an increase in the input enable range of the AD converter 34. On the other hand, in the present embodiment in which the negative drift cancellation (correction of reference voltage Vref) is performed, the level difference between the signs F and G does not change as illustrated in FIG. 8B, the input enable range required for the AD converter 34 provided in the rear stage of the differential amplifier 33 can be minimized.

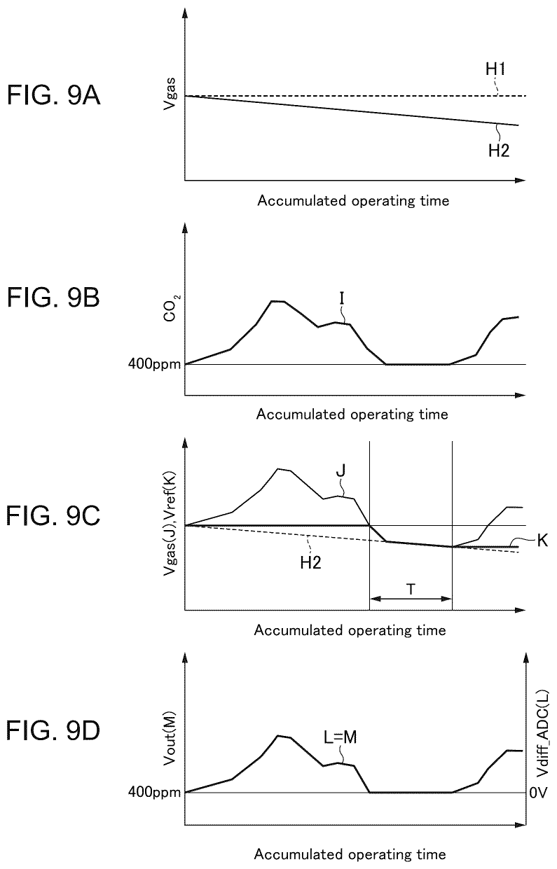

FIGS. 9A to 9D are schematic graphs for explaining an example of the operation of the gas sensor 100 when the concentration of CO₂ gas in measurement atmosphere changes. FIG. 9A illustrates a change in the drift amount of the gas detection signal Vgas, FIG. 9B illustrates a change in the actual concentration of CO₂ gas, FIG. 9C illustrates a change in the levels of the gas detection signal Vgas and reference voltage Vref, and FIG. 9D illustrates a change in the levels of the differential signal Vdiff_ADC and output signal Vout.

In FIG. 9A, a sign H1 denotes a correct level (level obtained when no drift is present) of the gas detection signal Vgas to be obtained when the concentration of CO₂ gas in measurement atmosphere is constant at a concentration value (e.g., 400 ppm) of CO₂ gas under ordinary atmospheric environment. In FIGS. 9A and 9C, a sign H2 denotes the level of the gas detection signal Vgas to be actually obtained when the concentration of CO₂ gas in measurement atmosphere is constant at a concentration value (e.g., 400 ppm) of CO₂ gas under ordinary atmospheric environment. Therefore, the difference between the signs H1 and H2 in FIG. 9A corresponds to the amount of a negative drift occurring in the sensor part 10.

In FIG. 9B, a sign I denotes the concentration of CO₂ gas in measurement atmosphere. In FIG. 9C, a sign J denotes the level of the gas detection signal Vgas to be actually obtained when the concentration of CO₂ gas in measurement atmosphere changes as illustrated in FIG. 9B, and a sign K denotes the level of the reference voltage Vref. In FIG. 9D, signs L an M denote the levels of differential signal Vdiff_ADC and output signal Vout to be actually obtained when the concentration of CO₂ gas in measurement atmosphere changes as illustrated in FIG. 9B.

Even when the concentration of CO₂ gas changes as illustrated in FIG. 9B, the concentration (e.g., 400 ppm) of CO₂ gas under ordinary atmospheric environment is a lower limit value, and the concentration of CO₂ gas does not fall below this value usually. However, when a negative drift has occurred in the sensor part 10, the level of the gas detection signal Vgas gradually becomes lower than a level corresponding to the actual concentration of CO₂ gas, as illustrated in FIG. 9C. Thus, when the actual concentration of CO₂ gas is lowered to around 400 ppm, the level of the gas detection signal Vgas is lowered to a level corresponding to less than 400 ppm. When such a state occurs, the reference voltage Vref is corrected such that the level thereof is lowered, as described using FIGS. 4 to 6. A sign T in FIG. 9C denotes a period of time during which the reference voltage Vref is corrected. As a result, as illustrated in FIG. 9D, the level of the differential signal Vdiff_ADC is corrected so as not to fall below a level corresponding to the concentration (e.g., 400 ppm) of CO₂ gas under ordinary atmospheric environment, and the output signal Vout to be finally output follows this.

As described above, in the present embodiment, the level of the reference voltage Vref is changed in accordance with the amount of a negative drift that has occurred in the sensor part 10, so that it is possible to obtain the output signal Vout without affecting the conversion operation from the differential signal Vdiff_ADC to output signal Vout performed by the control circuit 35. That is, the control circuit 35 generates the output signal Vout without correcting the differential signal Vdiff_ADC according to the reference voltage Vref, so that a calculation for conversion from the differential signal Vdiff_ADC to output signal Vout is not complicated.

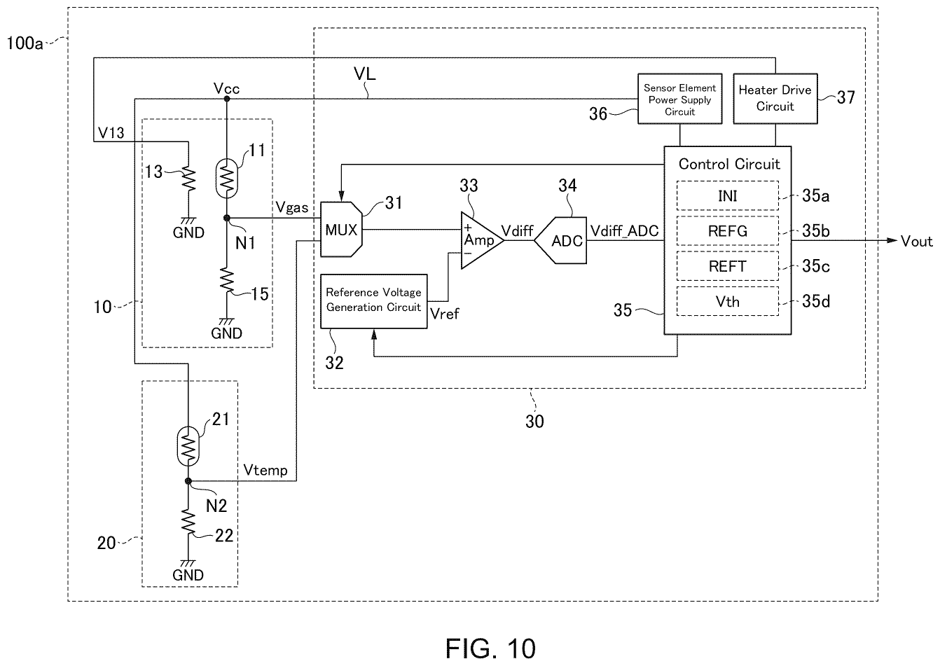

FIG. 10 is a circuit diagram illustrating the configuration of a gas sensor 100a according to a first modification.

As illustrated in FIG. 10, the gas sensor 100a according to the first modification differs from the gas sensor 100 illustrated in FIG. 1 in that a fixed resistor 15 is used in place of the thermistor 12 and that the heater 14 is omitted. Other basic configurations are the same as those of the gas sensor 100 illustrated in FIG. 1, so the same reference numerals are given to the same elements, and overlapping description will be omitted. As exemplified by the gas sensor 100a according to the first modification, the element on the reference side provided as a counterpart of the thermistor 11 for detection need not be a thermistor but may be a fixed resistor.

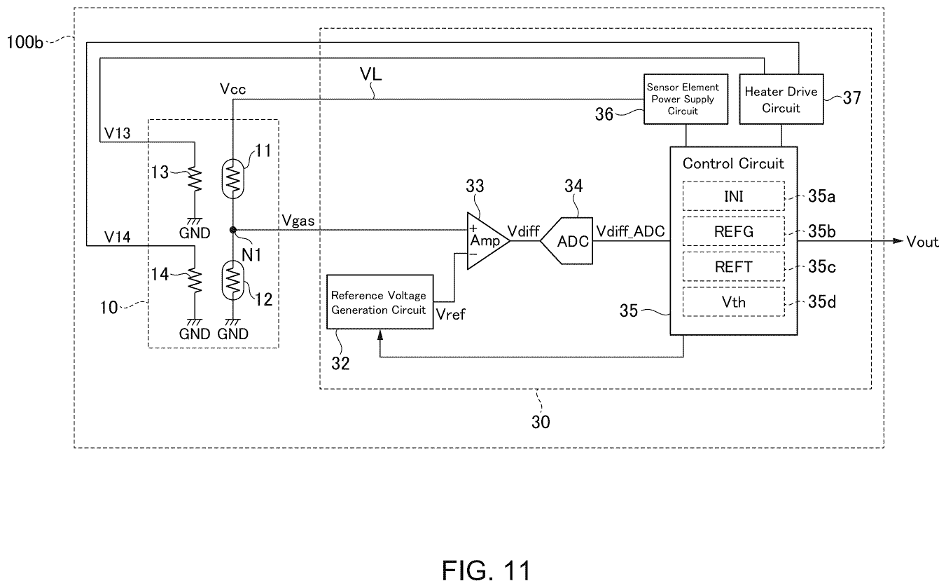

FIG. 11 is a circuit diagram illustrating the configuration of a gas sensor 100b according to a second modification.

As illustrated in FIG. 11, the gas sensor 100b according to the second modification differs from the gas sensor 100 illustrated in FIG. 1 in that the temperature sensor 20 and multiplexer 31 are omitted. Other basic configurations are the same as those of the gas sensor 100 illustrated in FIG. 1, so the same reference numerals are given to the same elements, and overlapping description will be omitted. As exemplified by the gas sensor 100b according to the second modification, the temperature sensor 20 need not necessarily be provided when the environmental temperature is retained substantially constant.

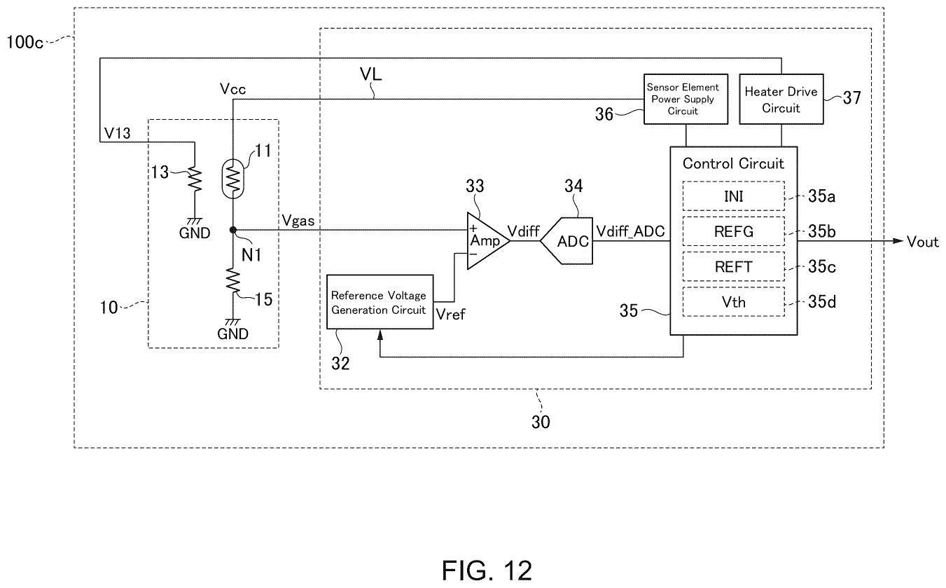

FIG. 12 is a circuit diagram illustrating the configuration of a gas sensor 100c according to a third modification.

As illustrated in FIG. 12, the gas sensor 100c according to the third modification differs from the gas sensor 100a illustrated in FIG. 10 in that the temperature sensor 20 and multiplexer 31 are omitted. Other basic configurations are the same as those of the gas sensor 100a illustrated in FIG. 10, so the same reference numerals are given to the same elements, and overlapping description will be omitted. As exemplified by the gas sensor 100c according to the third modification, the fixed resistor 15 may be adopted for the element on the reference side provided as a counterpart of the thermistor 11 for detection, and the temperature sensor 20 may be omitted.

While some embodiments of the technology according to the present disclosure have been described, the technology according to the present disclosure is not limited to the above embodiments, and various modifications may be made within the scope of the present disclosure, and all such modifications are included in the technology according to the present disclosure.

For example, although a thermistor, which is a resistor element, is used as the temperature-sensitive element of the sensor part 10 in the above embodiment, the present disclosure is not limited to this. For example, platinum (Pt) or tungsten (W), which is a resistor element, may be used as the temperature-sensitive element.

Further, although CO₂ gas is used as a gas to be measured in the above embodiments, the present disclosure is not limited to this. Further, the sensor part used in the present disclosure need not necessarily be a thermal conduction type sensor, but may be a sensor of other types such as a catalytic combustion type, a thermoelectric type, a semiconductor type, an electrochemical type, a solid-state type, or an optical type. As an example, when CO₂ gas is used as a gas to be measured, a catalytic combustion type sensor part can be used. In this case, since the concentration of CO₂ gas under normal conditions is substantially 0, the threshold value Vth may be set to a value of the differential signal Vdiff_ADC to be obtained when the concentration of CO₂ gas is 0.

The technology according to the present disclosure includes the following configuration examples, but not limited thereto.

A gas sensor according to an aspect of the present disclosure includes: a sensor part configured to generate a gas detection signal according to a concentration of a gas to be measured; a differential amplifier configured to amplify a difference between the gas detection signal and a reference voltage to generate a differential signal; and a control circuit configured to generate an output signal indicating the concentration of the gas to be measured based on the differential signal. When the concentration of the to be measured falls below a threshold value gas corresponding to a level of the differential signal that is determined to be a concentration of the gas to be measured under normal conditions, the control circuit is configured to correct the reference voltage such that a level of the difference signal becomes equal to or more than the threshold value. Thus, even when a negative drift has occurred in the sensor part, it can be canceled.

A gas sensor according to another aspect of the present disclosure includes: a sensor part configured to generate a gas detection signal according to a concentration of a gas to be measured; a differential amplifier configured to amplify a difference between the gas detection signal and a reference voltage to generate a differential signal; and a control circuit configured to generate an output signal indicating the concentration of the gas to be measured based on the differential signal. When the concentration of the gas to be measured falls below a threshold value corresponding to a level of the differential signal that is determined to be a concentration of the gas to be measured under normal conditions, the control circuit is configured to correct the reference voltage such that a level of the difference signal becomes equal to or more than the threshold value. Thus, even when a negative drift has occurred in the sensor part, it can be canceled.

In the above gas sensor, the control circuit may include a memory in which a set value concerning the reference voltage is updated when the reference voltage is corrected, and the control circuit may be configured to determine whether the difference signal falls below the threshold value in a state where the level of the reference voltage is set based on the set value stored in the memory. This allows the level of the reference voltage to follow the temporal change of a negative drift.

In the above gas sensor, the control circuit may be configured to generate the output signal without correcting the differential signal according to the reference voltage. Thus, a calculation of gas concentration based on the difference signal is not complicated.

In the above gas sensor, the control circuit may be configured to correct stepwise the reference voltage when the level of the differential signal falls below the threshold. This can suppress a calculation load on the control circuit.

In the above gas sensor, the control circuit may be configured to correct the reference voltage in accordance with the difference between the level of the differential signal and the threshold value when the level of the differential signal falls below the threshold value. This can correct the reference voltage at high speed.

In the above gas sensor, the gas to be measured may be CO₂ gas, and the concentration under normal conditions may be the concentration of CO₂ gas under ordinary atmospheric environment. Thus, there can be provided a CO₂ gas sensor capable of cancelling a negative drift.

What is claimed is:

1. A gas sensor comprising:
a sensor part configured to generate a gas detection signal according to a concentration of a gas to be measured;
a differential amplifier configured to amplify a difference between the gas detection signal and a reference voltage to generate a differential signal; and
a control circuit configured to generate an output signal indicating the concentration of the gas to be measured based on the differential signal,
wherein when the concentration of the gas to be measured falls below a threshold value corresponding to a level of the differential signal that is determined to be a concentration of the gas to be measured under normal conditions, the control circuit is configured to correct the reference voltage such that a level of the difference signal becomes equal to or more than the threshold value.

2. The gas sensor as claimed in claim 1,
wherein the control circuit includes a memory in which a set value concerning the reference voltage is updated when the reference voltage is corrected, and
the control circuit is configured to determine whether the difference signal falls below the threshold value in a state where the level of the reference voltage is set based on the set value stored in the memory.

3. The gas sensor as claimed in claim 1, wherein the control circuit is configured to generate the output signal without correcting the differential signal according to the reference voltage.

4. The gas sensor as claimed in claim 1, wherein the control circuit is configured to correct stepwise the reference voltage when the level of the differential signal falls below the threshold.

5. The gas sensor as claimed in claim 1, wherein the the control circuit is configured to correct the reference voltage in accordance with a difference between the level of the differential signal and the threshold value when the level of the differential signal falls below the threshold value.

6. The gas sensor as claimed in claim 1,
wherein the gas to be measured is CO₂ gas, and
the concentration under normal conditions is a concentration of CO₂ gas under ordinary atmospheric environment.

7. A gas sensor comprising:
a sensor part configured to generate a gas detection signal according to a concentration of a gas to be measured;
a differential amplifier configured to amplify a difference between the gas detection signal and a reference voltage to generate a differential signal; and
a control circuit configured to generate an output signal indicating the concentration of the gas to be measured based on the differential signal,
wherein when the concentration of the gas to be measured falls below a threshold value corresponding to a level of the differential that signal is determined to be a concentration of the gas to be measured under normal conditions, the control circuit is configured to correct the reference voltage such that a level of the difference signal becomes close to the threshold value.

8. The gas sensor as claimed in claim 7,
wherein the control circuit includes a memory in which a set value concerning the reference voltage is updated when the reference voltage is corrected, and
the control circuit is configured to determine whether the difference signal falls below the threshold value in a state where the level of the reference voltage is set based on the set value stored in the memory.

9. The gas sensor as claimed in claim 7, wherein the control circuit is configured to generate the output signal without correcting the differential signal according to the reference voltage.

10. The gas sensor as claimed in claim 7, wherein the control circuit is configured to correct stepwise the reference voltage when the level of the differential signal falls below the threshold.

11. The gas sensor as claimed in claim 7, wherein the the control circuit is configured to correct the reference voltage in accordance with a difference between the level of the differential signal and the threshold value when the level of the differential signal falls below the threshold value.

12. The gas sensor as claimed in claim 7,
wherein the gas to be measured is CO gas, and
the concentration under normal conditions is a concentration of CO gas under ordinary atmospheric environment.
